(19) United States
(12) Patent Application Publication
(10) Pub. No.: US 2015/0272440 A1
(43) Pub. Date: Oct. 1, 2015

(54) Tissue Tonometer and Associated Devices, Systems and Methods

(71) Applicants: Ruth Smith, Lamoni, IA (US); David McCormick, Prairie Village, KS (US); V. Craig Seyl, Olathe, KS (US)

(72) Inventors: Ruth Smith, Lamoni, IA (US); David McCormick, Prairie Village, KS (US); V. Craig Seyl, Olathe, KS (US)

(21) Appl. No.: 14/668,462

(22) Filed: Mar. 25, 2015

Related U.S. Application Data
(60) Provisional application No. 61/970,084, filed on Mar. 25, 2014.

(51) Int. Cl. A61B 5/00

(52) U.S. Cl. CPC A61B 5/0053; A61B 5/72; A61B 5/7425; A61B 5/442; A61B 5/4519

(57) ABSTRACT

A device for measuring tissue hardness of a subject comprising: a housing, the housing having a proximal end and a distal end; a contact probe configured to contact tissue arranged on the distal end of the housing; a sensor assembly configure to measure tissue hardness disposed within the housing; a microprocessor configured to receive input from the sensor assembly; a force transduction assembly disposed within the housing; a microprocessor receiving input from the displacement sensor and the force sensor; and a display, receiving input from the microprocessor, configured to display information to a user on the proximal end of the housing.

### CROSS-REFERENCE TO RELATED APPLICATION(S)

This application claims priority to U.S. Provisional Application No. 61/970,084, filed Mar. 25, 2014, which is hereby incorporated by reference in its entirety.

### FIELD OF THE INVENTION

The embodiments disclosed herein relate to various devices, systems, methods and related components, including systems and methods utilized in the identification and characterization of tissue tone.

### BACKGROUND OF THE INVENTION

Certain soft tissue conditions, which often result from tissue trauma or congenital disorders, can significantly inhibit physical function in various ways. These conditions involve a change in hardness of the tissue, causing pain, restricted motion, and/or compromised tissue health. In response, various medical and allied healthcare professionals are challenged to effectively treat these conditions. Unfortunately, current techniques to objectively measure the hardness of soft tissue are lacking in the medical field. The typical assessment depends on subjective palpation skills which are deemed highly unreliable.

Additionally, current treatment therapies to reduce tissue tension in muscle spasms and trigger points include direct manual pressure, commonly known as ischemic compression, a form of myofascial release. Although many clinicians have found this treatment to be beneficial, objective measures of its effectiveness are nonexistent. At this time, the clinician must rely solely on palpating the tissue's hardness and the patient's verbal response to elicit the desired reduction in the muscle tension. In other words, the clinician can only guesstimate the appropriate manual pressure and resulting effects of the treatment. Accordingly, there is a need in the art for a portable hand held device that can aid health care professionals in objectively measuring changes in tissue tension resulting from therapeutic interventions. The disclosed embodiments relate to a device, system and method for measuring tissue hardness in a subject.

### BRIEF SUMMARY OF THE INVENTION

Discussed herein are various embodiments relating to devices, systems and methods for measuring tissue hardness of a subject. In various descriptions, and for brevity, these embodiments may be described as a “tonometer,” though the use of that term is in no way meant to limit the description to a specific modality. In certain implementations the tonometer comprises an elongate housing further comprising a proximal end and a distal end; a probe assembly extending from the distal end of the housing configured to contact tissue; a sliding assembly contained within the housing fixedly attached to the probe assembly; a displacement sensor in mechanical communication with the sliding assembly to measure tissue hardness; and a force transduction assembly, configured to measure force applied by the user.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a side view of the tonometer device according to certain embodiments.

FIG. 2 shows a side view of the tonometer device according to certain embodiments with the housing removed.

FIG. 3 shows a side view of the sliding assembly.

FIG. 4 shows a side view of the probe assembly according to certain embodiments.

FIGS. 5 A and B show side views of various contact stops according to certain embodiments.

FIGS. 6A-C show side views of various contact probes according to certain embodiments.

FIG. 7 shows an image of the tomometer with various contact stops and contact probes according to certain embodiments.

### DETAILED DESCRIPTION

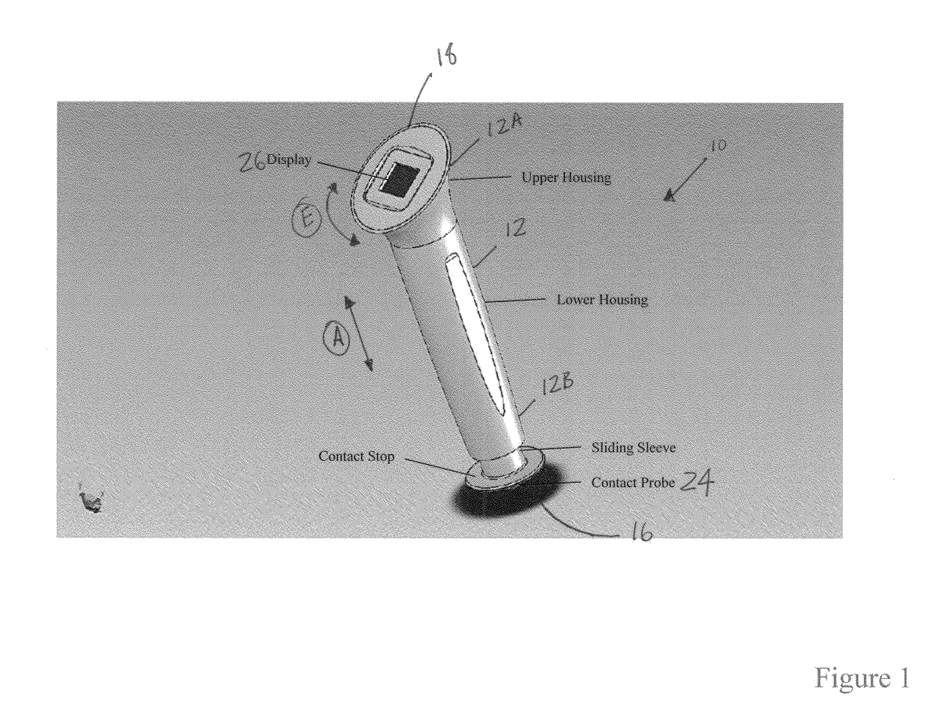

As is depicted generally in FIGS. 1-5, in certain exemplary embodiments, the soft tissue tonometer 10 is comprised of an elongate hand-held housing 12, and an internal functional structure or sensor assembly 14 configured to assess the tissue tone of a subject, and comprises distal 16 and proximal 18 ends. In certain embodiments, the tonometer further comprises a force sensor 20 and a displacement sensor 22. As a wireless unit, it is constructed with a distal contact probe 24 and proximal visual display 26. Further implementations feature a plurality of telescoping elements, such as an outer sleeve 28 and an inner sleeve 30, as well as a sleeve collar 32, and various associated components, such as a contact stop 34. These embodiments may well also feature a sensor arm 36, a microcontroller 38, and other associated components, such as a sensor cap 40.

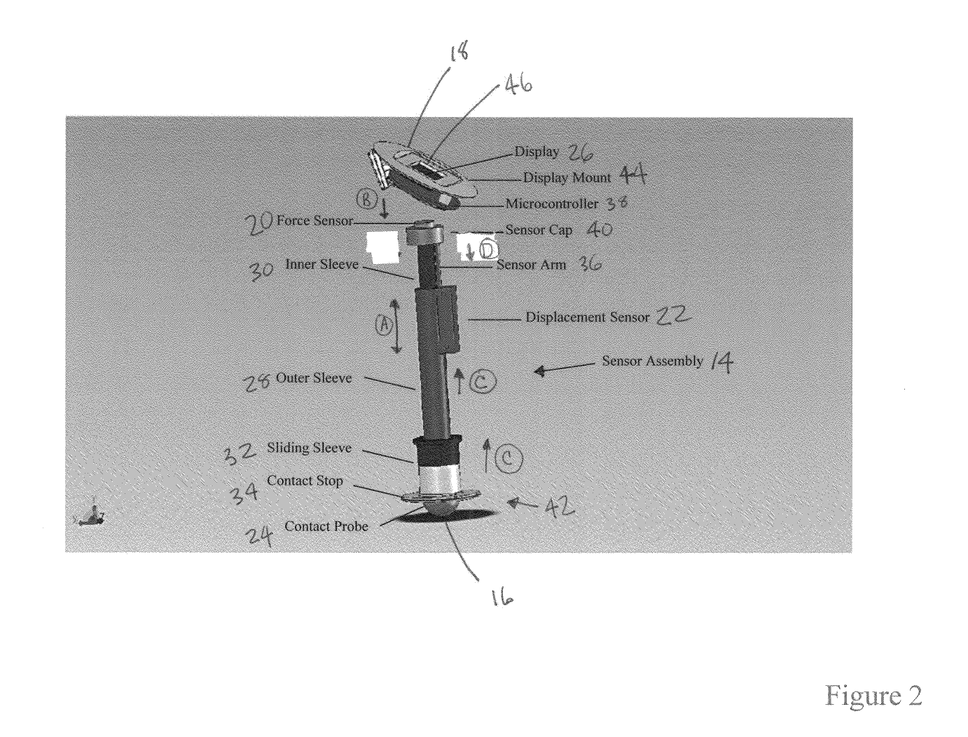

Turning now to the drawings in detail, FIG. 1 depicts an external view of the tonometer 10 according to an exemplary embodiment. In these implementations, the tonometer 10 comprises a generally elongate outer housing 12 further comprising an upper housing 12A and a lower housing 12B, all of which are substantially rounded and hollow, so as to accommodate the internal components discussed herein in relation to FIG. 2. As would be apparent to one of skill in the art, other configurations are possible. As shown in FIG. 1, these embodiments further comprise a contact probe 24 disposed near the distal end 16, and a display 26 disposed proximally 18. In certain embodiments, the housing defines and axis (designated by reference letter A) defined in relation to the central axis of the housing and extending toward the proximal 18 and distal 16 ends.

In operation, the contact probe 24 of the device is directly applied on the skin so as to measure the hardness of the tissue beneath it (or in certain embodiments the skin itself), which is also known herein as an “application.” For purposes of the description, the tonometer will be discussed as being applied to a subject by a user, though in certain applications the subject and user can be the same person. While positioning the tonometer 10 for application to the subject's tissue, the user applies the device such that the axis A is substantially perpendicular to the plane defined by the subject's tissue as would be understood by one of skill in the art. In these implementations, the user applies a force through the contact probe 24 to the target tissue.

In exemplary embodiments, the tonometer 10 features two sensors, the force sensor 20 and displacement sensor 22, which enable the tonometer to assess the force applied by the user by way of the force sensor, and the corresponding displacement of the subject's tissue by way of the displacement sensor) over time. In certain embodiments, the user must note the tissue hardness at the specified force in order to compare subsequent readings. For manual pressure therapy according to certain embodiments, the tonometer 10 provides constant feedback throughout an application. Accordingly, the user can adjust the amount of applied force to elicit a greater reduction in muscle tension of the spasm or trigger point. As the user observes the effect of the force, he/she can determine if more, less, or the same pressure should be continued. The user will attempt to maintain the effective amount of manual pressure until the hardness reading indicates an acceptable reduction in muscle tension.

According to certain implementations, the device calculates the tissue hardness relative to the applied force at the contact probe 24 and the distance traveled by the outer sleeve. As the device is pressed against the skin surface, the contact probe 24 will move into the skin as the outer sleeve remains at its original position on the surface. The harder the tissue, the more force is required to move the probe and the less distance traveled by the outer sleeve 28. In this way, tissue hardness is directly related to applied force and indirectly related to distance traveled.

According to certain embodiments, the outer housing 12 is constructed of a durable plastic material. In certain further embodiments the housing 12 is constructed from a durable polymer, composite, carbon composite, or metal materials, for example a metal or metal alloy such as aluminum or stainless steel. One skilled in the art will appreciate other materials are possible. In certain embodiments, the housing is comprised of an upper housing 12A and a lower housing 12B. According to certain embodiments, the upper housing 12A houses the display components and a microprocessor controller 38. In certain embodiments, the housing 12 has one or more mode control switches for selecting the operational mode of the device. According to certain implementations, the display is configured to display to the user information about the force applied by the user and/or the density of the tissue to which the force is applied. In certain embodiments the display displays information about the operational state of the device, such as operational mode, or power source charge state.

According to certain embodiments, the upper housing is rotatably coupled to the lower housing. In these embodiments, the user can rotate the upper housing 12B, as indicated by reference arrow E, such that the display can be easily viewed regardless of user position relative to the device.

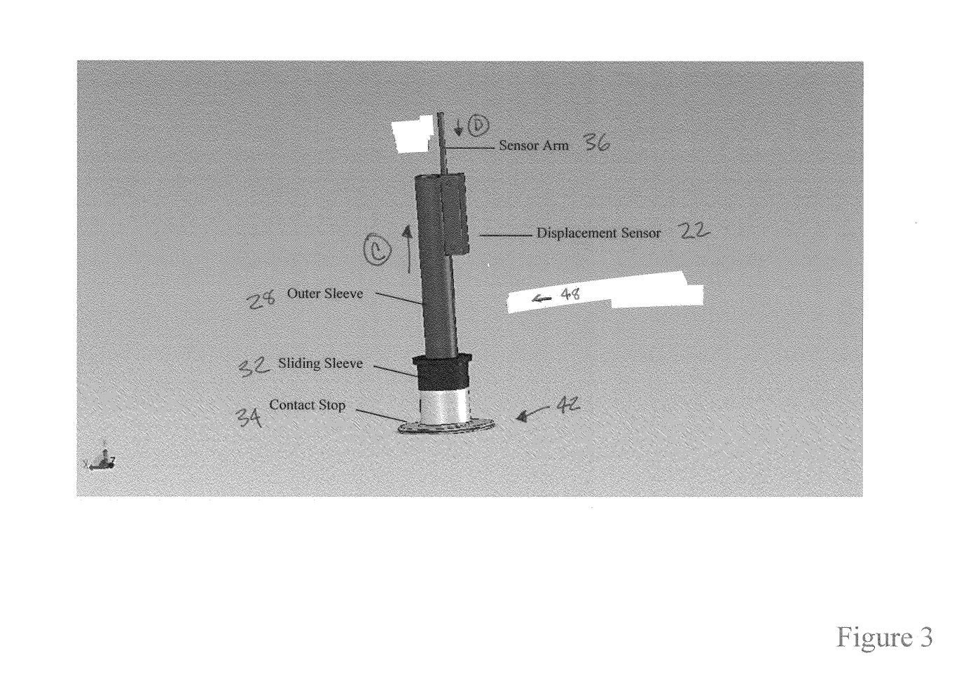

According to certain embodiments, disposed within the lower housing are a force sensor 20 that measures the force applied by the user to the tonometer 10 and a displacement sensor 22 that measures the distance of travel of the sensor assembly 14 (all of which are depicted herein in relation to FIGS. 2-3). According to certain embodiments, a sleeve collar 32 extends from the lower housing and is fixedly connected to a contact stop. In certain implementations a contact probe 24 extends from the contact stop 34 and is configured to contact tissue.

FIG. 2 shows an internal side view of the tonometer 10 according to certain embodiments wherein the outer housing has been 12 removed. In certain implementations, a force sensor 20 further comprising a sensor cap 40, disposed above an inner sleeve 30, which is capable of telescopic movement relative to the outer sleeve 28 in response to pressure exerted by the user at the proximal end 18. This movement, or compression (shown by reference letter B) is recorded or otherwise observed by the force sensor 20. In certain embodiments, the tonometer further comprises a displacement sensor 22 and a sensor arm 36, which is in mechanical communication the sensor cap 40 so as to move in a manner corresponding to the upward movement of the inner sleeve 30 in response to the pressure returned to the tonometer by the subject's tissue (designated by the reference letter C).

According to certain embodiments, a sensor assembly 14 is comprised of a substantially tubular outer sleeve 28, fixedly connected to a sleeve collar. According to certain implementations, a displacement sensor is mounted to the lateral surface of the proximal end of the outer sleeve. The displacement sensor further comprises a sensor arm that is operationally connected to a sensor cap. According to certain embodiments, the sleeve collar assembly is configured to receive the probe assembly. The probe assembly 17 comprises an inner sleeve fixedly connected to the sensor cap at its proximal end and the contact probe at its distal end. In certain embodiments, the force sensor 40 is mounted on the proximal surface of the sensor cap 40.

FIG. 2 shows the display assembly 26 comprising a display mount 44 and a display screen 46 according to certain embodiments. In certain embodiments, the display screen 46 is an LCD display. Disposed beneath the display assembly is a microcontroller configured to process data from the sensors and output information to the user via the display and/or to a PC via a USB connection. In certain embodiments the microcontroller manages power use by the device as described further below.

In certain embodiments, the display 26 is configured to display data parameters to the user gathered by the tonometer 10 during use. Examples of displayed data include, but are not limited to, force applied by the user, distance traveled by the sliding assembly, and calculated tissue density. In certain embodiments, parameters can optionally be displayed individually or simultaneously. In further embodiments, data parameters are displayed graphically so as to convey to the user, for example, the change in tissue density over time.

According to certain implementations, the display 26 is configured to signal to the user when optimal tissue depth has been reached. In further embodiments, the display 26 is configured to signal to a user when a maximal tissue depth is reached. In still further embodiments an indicator light arranged on the housing signals to the user when the optimal and/or maximal tissue depth is reached.

FIG. 3 shows a side view of the distal portion of the sensor assembly, or sliding assembly 48 according to certain embodiments. The outer sleeve 28 is fixedly connected to the sleeve collar 32. The sleeve collar 32 extends out from the base of the lower housing 12B (best seen in FIG. 1) and is fixedly connected to the contact stop 34. The contact stop 34 is configured to contact the tissue with a generally large flat surface area relative to the smaller rounded surface area of the contact probe 24. Upon the application of force, this larger flat contact area of the contact stop urges the sliding assembly to slide back in proximal direction towards the user while the contact probe 24 is pushed into the tissue. As the sleeve collar assembly is urged proximally (as is shown by reference arrow C), the sensor arm 36 is accordingly compressed between the displacement sensor 22 and the sensor cap (as shown by reference arrow D). The amount of compression is measured by the displacement sensor 22 to determine the amount that the sliding assembly has moved, which can be translated into an evaluation of compressive force from the probe assembly 17. In certain embodiments, the sliding assembly is tensioned by a spring.

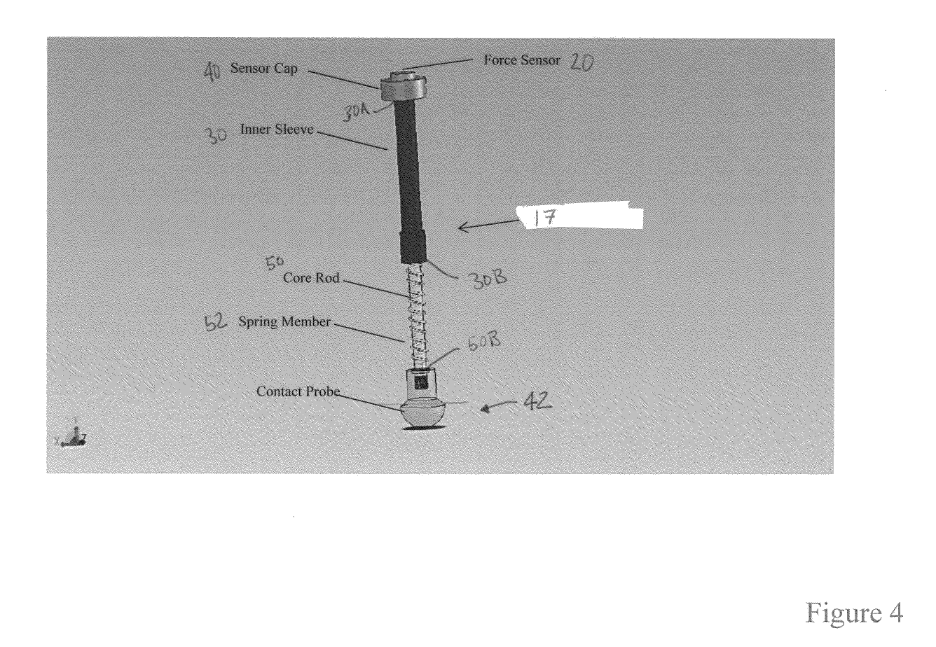

FIG. 4 shows a side view of the probe assembly 17 of the tonometer 10 according to certain embodiments. In these embodiments, the proximal end 30A of the inner sleeve 30 is fixedly connected to the sensor cap 40. A core rod 50 is telescopically is disposed within the inner sleeve 30, extending out of the distal end 30B of the inner sleeve. The distal end 50B of the core rod is fixedly connected to the contact probe 24 assembly. According to certain embodiments, a spring member 52 is operationally coupled the core rod 50 and displaced between the sensor cap 40 and contact probe 24 to bias the tonometer, such that it is at a resting state and able to be compressed under force, as would be understood by one of skill in the art.

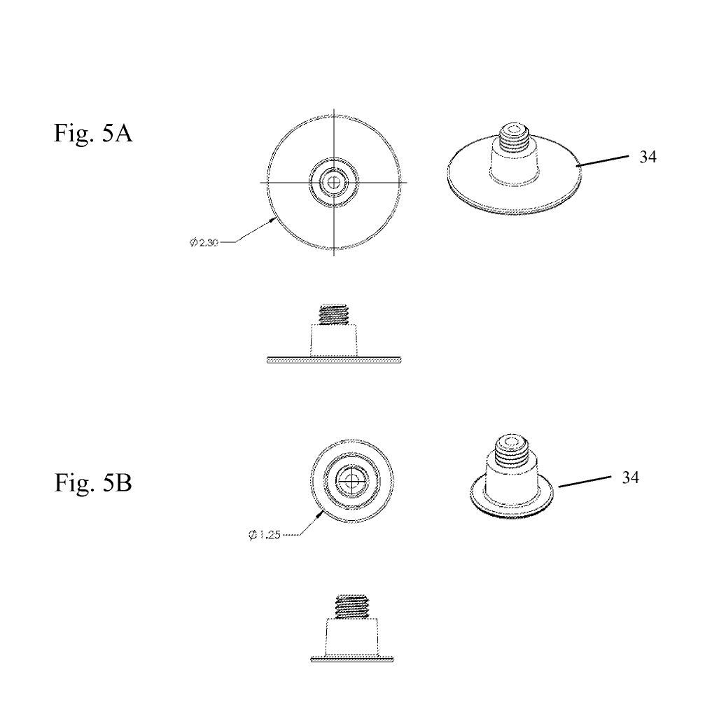

According to certain embodiments, best seen in FIGS. 5A and B, the contact stop 34 is removable. In certain further embodiments, the contact stop 34 is interchangeable with a plurality of the other contact stops. In certain implementations, the plurality of contact stops 34 vary in diameter. Different contact stop diameters can be selected based on the tissue to be sampled. For example, if a the surface area of the tissue to be sampled is large, such as a muscle in the back, the a contact stop with a large diameter is selected. If the tissue to be sampled is small, such as a muscle in the hand or face, then a contact tip with a small diameter is selected.

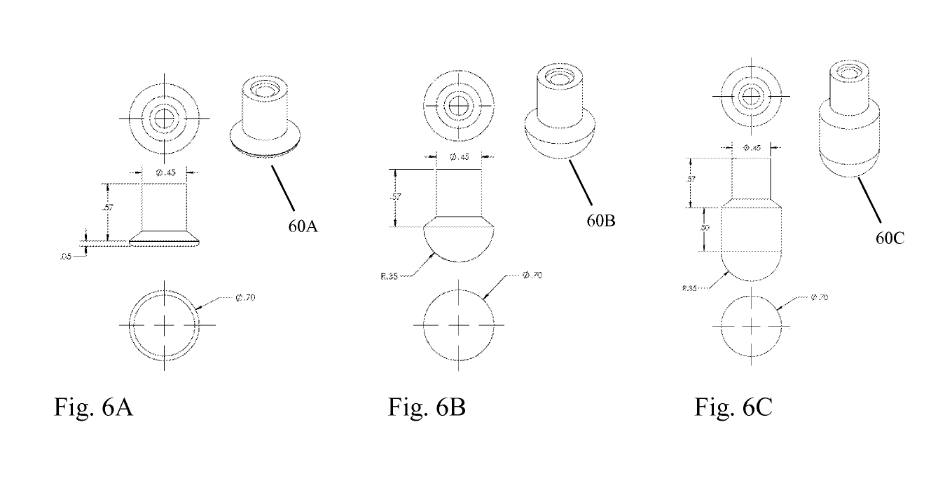

According to certain alternative embodiments best seen in FIGS. 6A and B, the contact probe 24 is removable. In certain further embodiments, the contact probe is interchangeable with a plurality of the other contact probes. In certain implementations, the plurality of contact probes vary in diameter and length. In these embodiments, a contact probe is selected according to tissue to be sampled or tissue depth to be sampled. In exemplary embodiments, if the user wishes to target a muscle spasm that is below a thick layer of skin/fat, the contact probe's tip 60 is selected that extends substantially beyond the contact stop because there is only a limited distance that the sleeve can travel in a proximal direction before the sensor arm is maximally compressed. By adjusting the length of protrusion of the contact tip 60 from the contact stop 34, the starting point is already a substantial distance below the skin's surface before any measurement is taken. Conversely, a contact tip 60 extending substantially beyond the contact stop would not be suitable for measurement of superficial tissue because the tip will not be able to penetrate far enough into the tissue for the contact stop to urge the contact stop in a proximal direction by way of the movement of the outer sleeve 28 relative to the inner sleeve 30.

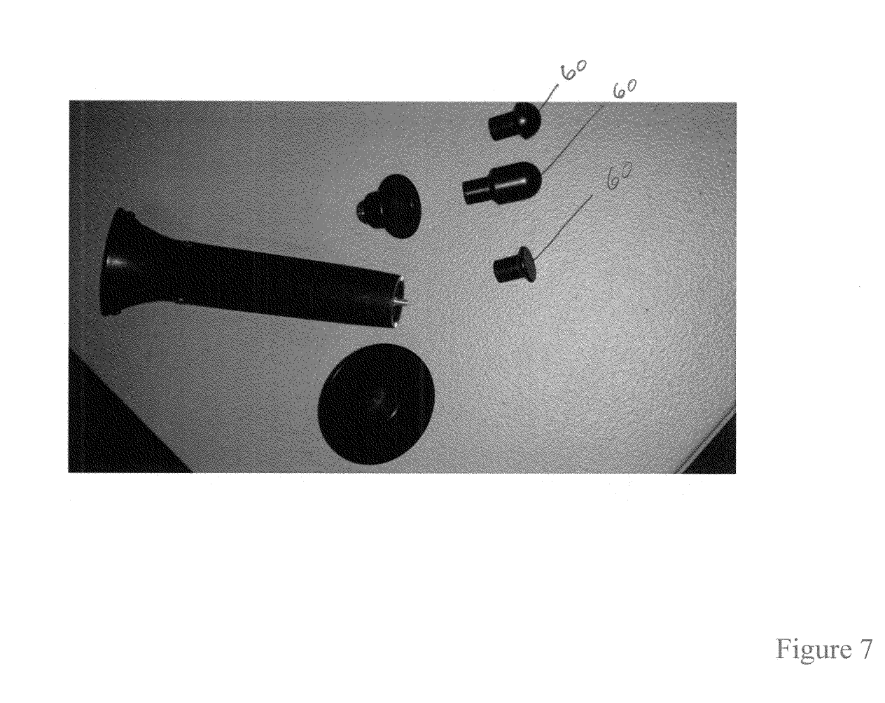

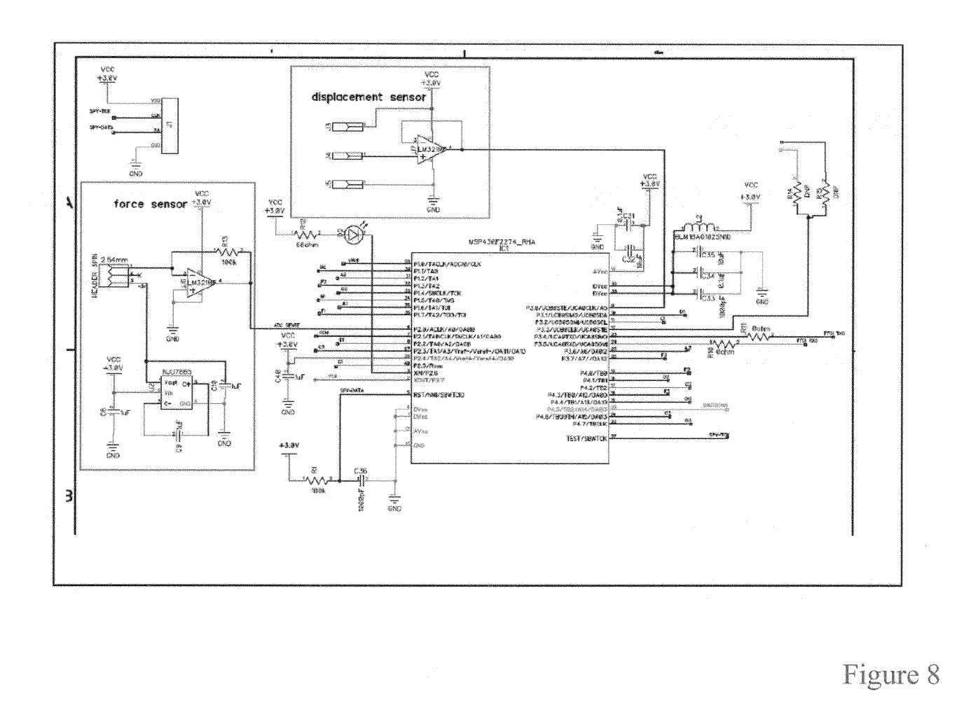

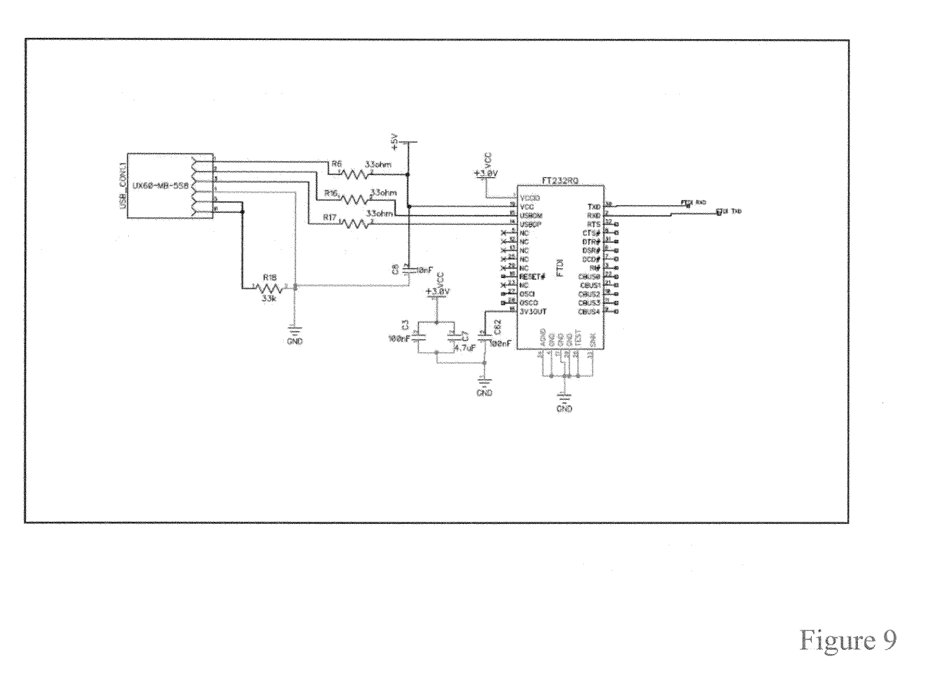

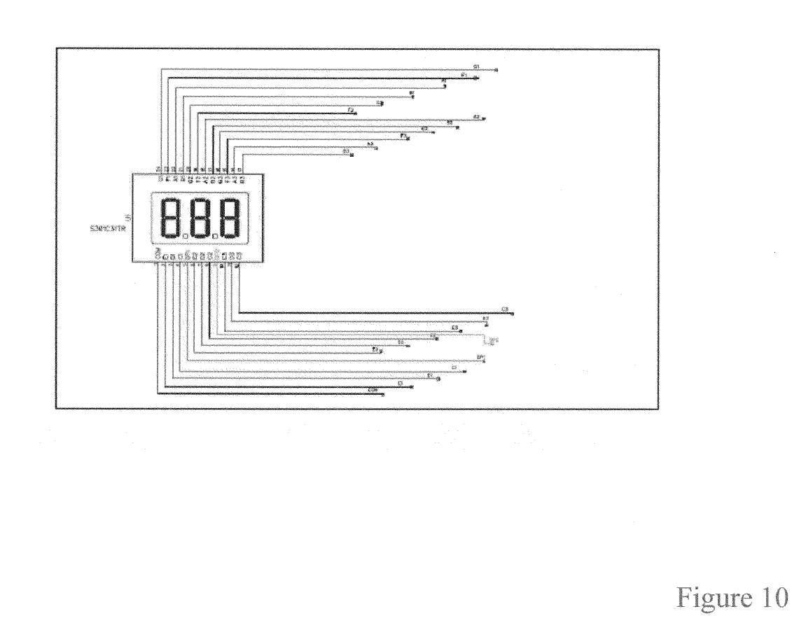

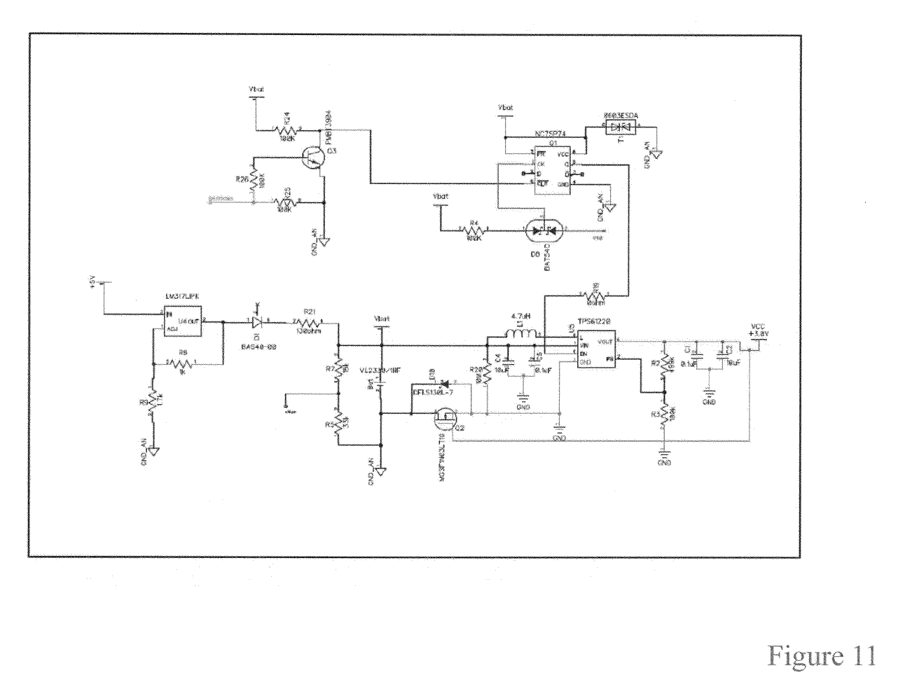

In certain exemplary embodiments, if skin is intended to be sampled, then a contact tip 60A which is substantially flush with the contact stop (as shown in FIG. 6A), or only barely protrudes from the contact stop, is selected. If superficial muscle is to be sampled, then a contact tip 60B is selected that extends approximately ½ inch from the contact stop, best shown in FIG. 6B. If deep muscle is to be sampled then a contact probe 60C that extends approximately 1 inch from the contact stop in FIG. 6C. One skilled in the art will recognize that a range of contact tips sizes can be selected and that the foregoing example tip sizes should not be viewed a limiting. A variety of such tips 60 are depicted in FIG. 7.

In certain embodiments the sensor sampling rate of the device measurement system is approximately 10 msec. According to certain embodiments, an on-board A/D converter is used for sampling. An on-board digitally controlled oscillator operates at a frequency of about 1 MHz. In certain implementations, sensor output is processed using op-amps. As will be appreciated by one skilled in the art, a variety of microprocessors/microcontroller can be used. In certain embodiments the microcontroller is a MSP430F2274. According to certain embodiments, the device enters a low power mode between measurements.

According to certain embodiments, the tonometer has circuitry underlying the USB connection capacity. In certain embodiments, the CTS is grounded and no other handshaking is required.

In certain embodiments, the display is driven at 80 Hz, directly from the controller. According to further embodiments, DNP resistors determine the decimal point used on the display.

According to certain alternative embodiments, the tonometer is configured to communicate wirelessly with one or more external device. In exemplary embodiments, the external device can be for example, an auxiliary display, mobile device, personal computer, or cloud based server. As used herein a “mobile device” can comprise any of a wide variety of devices, such as, without limitation, a mobile phone, smartphone, personal digital assistant, tablet computer, handheld computing device, smart watch and the like. Exemplary mobile devices can include or consist of mobile devices that run the Android™, IOS, or Windows Mobile operating system (or some subset thereof). As will be recognized, however, the device disclosed herein may be used with other mobile device operating systems, including operating systems that may be developed in the future. In these embodiments, the tonometer can output data generated during use of the device to the external device for analysis of data after use or real time display of data during use. In certain embodiments, the tonometer display may be deactivated during data display on an external device to conserve battery power of the tonometer. In further embodiments, the tonometer display be omitted from the device entirely and the user views device data exclusively through the use of the external device.

According to certain further embodiments, the device further comprises one or more memory systems for storing data generated during use of the device. Disclosed memory systems typically reside in one or some combination of computer usable volatile memory, e.g. random access memory (RAM), and data storage unit. However, it is appreciated that in some embodiments, operating system may be stored in other locations such as on a network or on a flash drive; and that further, operating system may be accessed from a remote location via, for example, a coupling to the internet or a mobile device. In one embodiment, for example, data is stored as an application or module in memory locations within RAM and memory areas within a data storage unit.

In certain implementations, the device further comprises a power supply. In certain implementations, the power supply turns off by the controller automatically after two minutes of no use as determined by changing A/D values. In certain implementations, the power supply is turned on when the user presses a button located on the housing. In still further embodiments, the power supply can be charged via USB connection. In yet further embodiments, power supply voltage is monitored using Vmon. According to certain embodiments, when the controller detects that the power supply is low, it will cause the display to display an indicator signal. In certain exemplary embodiments, a low power status is indicated by the a display of “BAT” on the display.

In certain aspects, further disclosed is a method of reducing tension in the muscle, or other tissue, of a subject comprising contacting the muscle of a subject with a tissue tonometer device, the device being capable measuring and displaying muscle hardness and force applied by user; reading an initial muscle tension level from the device; applying pressure to the muscle with the device and reading subsequent muscle tension levels until muscle tension has decreased from the initial muscle tension level by a predetermined amount.

Although the present invention has been described with reference to preferred embodiments, persons skilled in the art will recognize that changes may be made in form and detail without departing from the spirit and scope of the invention.

What is claimed is:

1. A device for measuring tissue hardness of a subject, the device comprising:
a. an elongate housing further comprising a proximal end and a distal end;
b. a probe assembly extending from the distal end of the housing configured to contact tissue;
c. a sliding assembly contained within the housing fixedly attached to the probe assembly;
d. a displacement sensor in mechanical communication with the sliding assembly to measure tissue hardness; and
e. a force transduction assembly, configured to measure force applied by the user.

2. The device of claim 1, wherein the sliding assembly further comprises an outer sleeve, a sleeve collar fixedly connected to the outer sleeve, and a contact stop extending laterally from the sleeve collar and configured to contact the tissue of the subject.

3. The device of claim 2 wherein the probe assembly further comprises a contact probe.

4. The device of claim 3, wherein the contact probe is removably coupled to the probe assembly.

5. The device of claim 4, further comprising an interchangeable contact probes, which is removably coupled to the probe assembly.

6. The device of claim 5, further comprising a plurality of removably coupleable contact probes, wherein at least one of the plurality of interchangeable contact probes is substantially flush with the contact stop when removably fixed to the probe assembly and wherein at least one of the plurality of contact probes extends distally from the contact stop when removably connected from the probe assembly.

7. The device of claim 3 wherein the probe assembly further comprises a inner sleeve and a core rod telescopically disposed within the inner sleeve, said core rod operationally connected to the contact probe.

8. The device of claim 7, further comprising a sensor cap fixedly connected to the inner sleeve.

9. The device of claim 8, wherein the sensor assembly further comprising a displacement sensor.

10. The device of claim 9, wherein the sensor assembly further comprises a sensor arm having a first end and a second end, wherein the sensor arm is operationally coupled to the sensor cap at the first end and operationally coupled to the displacement sensor at the second end.

11. The device of claim 8, further comprising a force sensor arranged on the sensor cap, configured to measure force applied by the user.

12. The device of claim 11, further comprising a first microprocessor configured to receive input from the sensor assembly and a second microprocessor receiving input from the displacement sensor and the force sensor.

13. The device of claim 1 further comprising a display arranged on the proximal end of the housing, receiving input from the microprocessor, configured to output data to a user.

14. The device of claim 12, wherein the housing further comprises an upper housing and a lower housing and the display is arranged within the upper housing.

15. The device of claim 14 wherein the upper housing is rotatably connected to the lower housing and adapted to be rotated by the user to view the display.

16. The device of claim 12, wherein the display is configured to display one or more of force applied by user, tissue hardness, and distance traveled by the sliding assembly.

17. The device of claim 1, further comprising an indicator signal, configured to signal the user when optimal tissue depth is reached.

18. The device of claim 17, wherein the indicator signal is configured to signal the user when maximal tissue depth is reach.

19. A tonometer system for measuring the firmness of subject skin tone, comprising:
a. an elongate housing further comprising a proximal end and a distal end;
b. a probe assembly extending from the distal end of the housing configured to contact tissue;
c. a sliding assembly contained within the housing fixedly attached to the probe assembly;
d. a displacement sensor in mechanical communication with the sliding assembly to measure tissue hardness;
e. a force transduction assembly, configured to measure force applied by the user; and
f. a display in electrical communication with the displacement sensor and force transduction assembly;
wherein the tonometer is configured such that the probe assembly may be depressed against a subject's skin and the amount of force applied to and received from the subject's skin can be displayed to monitor tissue density.

20. A method of reducing tension in the muscle of a subject comprising:
a. contacting the muscle of a subject with a tissue tonometer device, the device being capable measuring and displaying muscle hardness and force applied by user;
b. reading an initial muscle tension level from the device;
c. applying pressure to the muscle with the device and reading subsequent muscle tension levels until muscle tension has decreased from the initial muscle tension level by a predetermined amount.
